(19) United States
(12) Patent Application Publication
(10) Pub. No.: US 2003/0216632 A1
(43) Pub. Date: Nov. 20, 2003

(54) Ferromagnetic sensing method and apparatus

(75) Inventors: Richard J. McClure, San Diego, CA (US); R. Kemp Massengill, Leucadia, CA (US); William F. Avrin, San Diego, CA (US)

Correspondence Address: GERALD W SPINKS, P. O. BOX 2330, PORT ORCHARD, WA, 98366, US

(73) Assignee: MedNovus, Inc.

(21) Appl. No.: 10/354,905

(22) Filed: Jan. 29, 2003

Related U.S. Application Data
(63) Continuation-in-part of application No. 10/244,109, filed on Sep. 12, 2002.
(63) Continuation-in-part of application No. 10/017,913, filed on Oct. 29, 2001.
(60) Provisional application No. 60/428,606, filed on Nov. 22, 2002.
(60) Provisional application No. 60/385,056, filed on May 31, 2002.
(60) Provisional application No. 60/366,799, filed on Mar. 22, 2002.

(51) Int. Cl. A61B 5/05

(52) U.S. Cl. 600/409

(57) ABSTRACT

A method and apparatus for sequentially exposing a region of a patient to a low level DC magnetic field, first without movement of the region to null the sensing apparatus, then with movement of the region to detect synchronous changes in the detected signal, indicative of a ferromagnetic foreign body in the region. The nulling and signal detection can take place at a plurality of test zone set points at increasing magnitudes of the applied magnetic field. The sensing apparatus can be nulled at each test zone set point prior to introduction of the patient.

### CROSS REFERENCE TO RELATED APPLICATIONS

This is a continuation-in-part application of copending U.S. pat. app. Ser. No. 10/244,109, filed on Sep. 12, 2002, for “Pre-Screening Utilizing Magnetic Resonance Imaging Field”; and a continuation-in-part of copending U.S. pat. app. Ser. No. 10/017,913, filed on Oct. 29, 2001, for “Ferromagnetic Foreign Body Detection Utilizing Eye Movement”. This application claims the benefit of U.S. Provisional Pat. App. No. 60/428,606, filed on Nov. 22, 2002, and entitled “Ferromagnetic Sensing Method and Apparatus”; U.S. Provisional Pat. App. No. 60/385,056, filed on May 31, 2002, and entitled “Pre-Screening Utilizing Magnetic Resonance Imaging Field”; and U.S. Provisional Pat. App. No. 60/366,799, filed on Mar. 22, 2002, and entitled “Pre-Screening Utilizing Magnetic Resonance Imaging Field”.

### STATEMENT REGARDING FEDERALLY SPONSORED RESEARCH OR DEVELOPMENT

Not Applicable

### BACKGROUND OF THE INVENTION

1. Field of the Invention

The present invention is in the field of human magnetic resonance imaging.

2. Background Art

Patients requiring magnetic resonance imaging (MRI) studies will obligatorily be exposed to very large magnetic fields. These magnetic fields are of sufficient size to cause motion of magnetizable particles in the body.

The body area most susceptible to damage is the eye, because of the general absence within the eye of constraining structures. Severe intraocular damage has been documented in MRI procedures, including irreversible loss of vision. Another area where severe damage can occur is the brain, and ferromagnetic particles, such as clips from neurosurgical procedures, can move when the large field associated with magnetic resonance imaging is applied, thereby wreaking havoc within brain tissue. Although neurosurgical clips in present use are generally manufactured from non-magnetic materials, in the past, clips were typically ferromagnetic in nature.

The most devastating effect of a ferromagnetic foreign body (FFB) within the eye, the orbit, or the brain is the torque effect induced by the MRT magnetic field upon the ferromagnetic object. Objects which are spherical do not rotate in the MRI magnetic field; however, most objects are not spherical. Therefore, most objects will tend to align with the magnetic field. For an elongated ferromagnetic foreign body, this alignment is akin to a “propeller” effect, which can result in cutting through ocular or neurological tissues.

In most areas of the body, although damage can occur, the damage tends to be less extensive, because the body forms scar tissues and essentially encapsulates the ferromagnetic foreign body. This encapsulation does not occur to any significant degree within the eye, the orbit, or the brain. The main areas of risk for MRI scanning, then, are the eye, the orbit, and the brain. The eye can have retained ferromagnetic foreign bodies which are subjected to forces or torque which pull or rotate the object through the ocular tissues, which may cause a permanent loss of vision. Obviously, welders and machinists are at particularly high risk. The clinical literature contains case studies of patients with previously unknown intraocular ferromagnetic foreign bodies suffering blindness during or after an MRI scan. Motion of the FFB can cause deleterious effects, such as vitreous hemorrhage, retinal damage, or damage to the optic nerve. A similar problem exists within the brain, since a retained intracranial ferromagnetic foreign body can be moved through brain tissue under the influence of the large magnetic field of the MRI instrument. Tragic neurological deficit may be a consequence.

The significance of the problems encountered is related to the large size of the magnetic field during magnetic resonance imaging. A typical MRI system generates a magnetic field strength between 10,000 Oersted (Oe) and 15,000 Oe, or 1 to 1.5 Tesla (T). The MRI field cannot be readily switched off, so the patient is inserted into the MRI machine with the magnetic field activated. During positioning of the patient within the MRI magnetic field, the patient and, therefore, a retained FFB, experience an increasing magnetic field, which may exert torque on the FFB, causing a rotational movement of the object, especially in the eye, the orbit, or the brain, and, in high magnetic field gradient regions, also a translational force, tending to move the particle if it is not encapsulated by scar tissue. A particularly acute potential problem exists in the eye, the orbit, or the brain, where tissues constraining movement are minimal. The magnetic field of the MRI system is very large, extending for a distance of several feet. Thus, for example, when the knee is scanned, a large magnetic field is applied not only to the knee, but also to the eye, to the orbit, and to the brain. Therefore, even during magnetic resonance imaging of the knee, torquing and/or translational movement of a retained FFB within the eye, orbit, or brain, can occur.

Damage can arise not only in conventional MRI units with magnetic field strengths of 1 Tesla or higher, but has also been documented in an MRI instrument with a field strength of 0.35 Tesla (3,500 Oe). Laboratory studies with freshly harvested animal and cadaver eyes, however, have found that intraocular and intraorbital FFBs show the most significant movement during an MRI procedure with larger fields, such as 1 to 1.5 Tesla. In the absence of a suitable and practical pre-MRI screening test, the consensus in the literature is that the risk of trauma from retained FFBs for metal workers is often sufficient to withhold magnetic resonance imaging, at least at magnetic field strengths greater than 0.35 Tesla.

The known presence of ferromagnetic foreign bodies in the eye, orbit, or brain, and sometimes elsewhere, is currently an established contraindication for MRI procedures. However, a patient often does not know when a foreign object is present, or the patient does not provide such information to the radiologist or technician. Therefore, radiologists performing MRI scanning may be unaware that a patient has a retained ferromagnetic foreign body in a critical body region, with potentially catastrophic consequences.

Risk determination associated with medical procedures is often difficult. Physicians must daily weigh the costs and risks of diagnostic procedures against the information gained, sometimes with inadequate information as to the probability of an adverse reaction. In the case of MRI, the availability of a low-cost, low-risk screening procedure to detect FFBs would allow physicians to make the correct decision in an informed way, thereby avoiding the risk of damage without depriving patients needlessly of a vital diagnostic modality.

X-ray radiography has shown mixed results in detecting foreign bodies within or near the eye. Although the technology provides generally excellent resolution of even tiny FFBs, the fact that an x-ray must be interpreted can lead to human error. CT procedures have certain advantages, but CT scans are too expensive for routine screening purposes. Both CT scans and X-ray radiography share another serious problem, in that these modalities can differentiate between metallic and nonmetallic foreign bodies, but not between ferromagnetic and non-ferromagnetic foreign bodies. If all patients having retained metallic objects, some of which were ferromagnetic, and some of which are not ferromagnetic, were denied the application of MRI scanning, a significant number of these patients would be needlessly deprived of a valuable diagnostic procedure. To base a clinical decision to perform, or not to perform, an MRI procedure upon plain X-ray films showing a metallic foreign body is undesirable, as the X-ray does not answer vital questions regarding the ferromagnetic nature of the retained metallic foreign object in question. This problem, together with the fact that ionizing radiation is inherent in radiological tests, and, additionally, the occasional failure because of human interpretational error to detect significant and potentially dangerous objects, are drawbacks inherent with X-ray and CT techniques for routine pre-MRI screening.

Ultrasound is another methodology which can be employed for detecting foreign bodies in or near the eye, or elsewhere. One study documented that ultrasound has a probability of detection, for all types of foreign body materials, of 93%. Ultrasound procedures suffer the same deficiency as X-ray and CT procedures, however, in that ultrasound cannot distinguish ferromagnetic from non-ferromagnetic foreign bodies.

Passive magnetic detection, as used in magnetoencephalography (MEG), can detect the perturbation of the earth's magnetic field caused by a ferromagnetic object. Algorithms exist which allow localization and characterization of an object in question by measuring a complete set of magnetic field and gradient tensor components. However, the ultra-sensitive magnetometers used for MEG are exceedingly expensive. Furthermore, the static field perturbations caused by an FFB can be difficult to distinguish from background fields.

It would be advantageous, then, to have a practical ferromagnetic foreign body detection system designed to screen patients before the commencement of magnetic resonance imaging. With the tremendously increasing popularity of magnetic resonance imaging, such screening could reduce the morbidity and human suffering caused by MRI-induced movement of ferromagnetic foreign bodies within, or adjacent to, vital organs.

### BRIEF SUMMARY OF THE INVENTION

It is an object of the present invention to provide a method and apparatus for the detection of ferromagnetic foreign bodies retained within a region of a patient before that patient undergoes magnetic resonance imaging. With appropriate software, the location and size of a retained ferromagnetic foreign body within the diagnostic region of interest of a patient can be determined. The present invention provides a noninvasive, safe, economical, and easy-to-use method and apparatus for detection, location, and characterization of a retained ferromagnetic foreign body.

In order to detect the magnetic field emanating from a ferromagnetic foreign body in a particular body region, such as within the eye, the orbit, or the head, it is necessary to apply a small magnetic field to that region to magnetize the FFB. The present invention, then, provides for a DC magnetic field source, including, but not limited to, a permanent magnet, or, alternatively, a coil with an appropriate power supply, or a magnetic core with external coil and power supply, to function as a convenient source of applied DC magnetic field, which is utilized to magnetize the foreign body and thus allow it to be sensed upon movement as described herein before subjecting the patient to the huge, and potentially dangerous, magnetic field within the MRI-measurement zone. Although the present invention can be utilized with appropriate configurations to pre-screen prior to magnetic resonance imaging other parts of the body, it is ideally suited for the eye.

The present invention comprises:

1. a method for detecting ferromagnetic foreign bodies in a sensed region of the body, such as the eye, the orbit, or the brain, wherein the foreign body is magnetized for detection by a DC magnetic field source and detected by the sensor system apparatus of the present invention.

2. an apparatus for detecting ferromagnetic foreign bodies in a sensed region of the body, such as the eye, the orbit, or the brain, wherein the foreign body is magnetized for detection by a DC magnetic field source and detected by the sensor system of the present invention.

The present invention is ideally suited for pre-screening the eye prior to magnetic resonance imaging, and, for clarity of documentation, the drawings herein will depict eye pre-MRI screening. Alternative configurations, however, can be constructed employing the method of the present invention for pre-MRI screening other parts of the body.

The novel features of this invention, as well as the invention itself, will be best understood from the attached drawings, taken along with the following description, in which similar reference characters refer to similar parts, and in which:

### BRIEF DESCRIPTION OF THE SEVERAL VIEWS OF THE DRAWINGS

FIG. 1 is a schematic diagram of the apparatus of the present invention;

FIG. 2 is a diagram of the magnetic field generated by the DC magnetic field source of the present invention;

FIG. 3 is a graph of the magnetic field strength at various distances from the source; and

FIGS. 4 through 9 show a portion of the apparatus of the present invention configured to make measurements at various distances from the source.

### DETAILED DESCRIPTION OF THE INVENTION

In U.S. pat. app. Ser. No. 10/244,109, the disclosure of which is incorporated herein by reference, provision was made for sensing a ferromagnetic foreign body (FFB) within a patient by utilizing the existing magnetic field region of a magnetic resonance imaging (MRI) instrument to induce magnetization of the FFB particle. Special emphasis was given to the eye, since this is a particularly vulnerable organ subject to damage from the magnetic field of an MRI machine, if a FFB is retained intraocularly.

It is not necessary, however, to use such an expensive and complex system as an MRI instrument to provide the necessary applied magnetic field. It may be necessary or convenient to have pre-MRI sensing performed at a separate time and in a remote location away from the MRI instrument.

The present invention, therefore, uses the field from a separate DC magnetic field source, such as a portable permanent magnet, to induce magnetization of the FFB. Although the present invention can be utilized with appropriate configurations to pre-screen prior to magnetic resonance imaging other parts of the body, it is also ideally suited for the eye.

It is practical to use for a DC magnetic field source a permanent magnet, such as a Neodymium/Iron/Boron permanent magnet, which, due its intrinsic properties, is relatively stable in a normal environment and has a large enough external magnetic field region to provide an adequate field strength without an excessive magnetic field gradient. Alternatively, a DC magnetic field source may be a provided by other means, including, but not limited to, a coil with suitable dimensions and power supply, or a magnetic core with external coils and power supply.

As in U.S. pat. app. Ser. No. 10/244,109, the sensor in the present invention is arranged in such a way as to have its surface perpendicular to the magnetic field of the DC magnetic field source, and the relative positions are adjusted along the axis of the magnetic field perpendicular to the surface of the DC magnetic field source to adjust the magnetic field strength to the desired magnitude.

Because the magnetic field diverges in both axes orthogonal to the longitudinal axis being used, it is necessary to make provision for small angular positional adjustments of the DC magnetic field source structure. Thus, this enables nulling of unwanted components of the applied magnetic field which may otherwise lie in the plane of the sensor.

In addition, adjustments of the applied magnetic field components may be used to null environmentally existing and unwanted components of DC magnetic fields which may also lie in the plane of the sensor. This may be accomplished using the main DC magnetic source, but more readily by the use of smaller, supplementary magnets, or other compensating ferromagnetic materials, such as coil apparati.

The sensor typically has a relatively narrow range of magnetic field sensing capability. In a readily available sensor comprising a Nickel/Iron (Permalloy) sensing layer, the linear range of operation is plus or minus 2 Oersted (Oe), and saturation occurs at or minus 10 Oe. For this reason, it is preferable to adjust the main DC magnetic field source as a factory adjustment and to employ for fine tuning smaller, supplementary magnets, or other compensating ferromagnetic materials, which provide magnetic fields of lesser magnitude, thereby avoiding problems arising from sensor saturation.

The sensors generally embody a reset provision. This provision enables the desirable, non-saturated sensing condition to be re-established automatically during use.

In operation, the goal is to detect the magnetic field emanating from quite small FFB particles, but provision is also made for detecting larger FFB particles. Therefore, controls are provided to enable compensation of large signals from external magnetic fields to be reduced to small values with coarse adjustments. This is followed by finer adjustments in the higher sensitivity range to allow detection of the tiny FFB particles.

Sensing registers on a visual display, which is monitored by the operator, and controls are utilized to bring the sensing indicator in the direction of a null. When a null has been achieved, the eye of the patient is placed in the sensing region, which is adjacent to the sensor.

In the event of the presence of a large FFB, an immediate displacement of the sensing indicator will be observed. In order to discern whether this is an artifact or a valid FFB, a new null may be re-established, and the next step in the procedure will be to require motion of the eye about horizontal and vertical axes in sequence upon instructions given to the patient. Deflection of the sensing indicator induced by eye movement provides evidence for the presence of an FFB within the eye.

If the patient has a retained FFB, such as shrapnel, in the vicinity of the eye (such as in the orbit or the skull), but not intraocularly, a deflection of the sensing indicator would be noted, but this deflection remains constant upon movement of the eye. The patient would be then reported for further investigation, such as x-ray radiography. On the other hand, an intraocular FFB would generate movement of the sensing indicator synchronous with eye movements.

In order to differentiate happenstance movements of the indicator due to variations in environmental magnetic fields from those emanating from a FFB within the eye, the patient is directed to follow a moving eye-tracked light-source icon, such as a light-emitting diode (LED), whose movement is dictated by an electrical signal which is registered on the display as a separate indication, thus enabling perception of synchronicity. Alternatively, verbal commands may be employed to induce eye movements. These verbal commands may be pre-recorded. Synchronous movements of the detected FFB signal with that of the eye-tracked light-source signal display provides strong indication of an intraocular FFB.

The procedure described above is then repeated for the fellow eye. To ensure cooperation of the patient, the operator observes eye motion of the paired eye. If the patient has only one eye, such as following enucleation for malignant tumor, the diligent operator will still be able to detect movement of the solitary eye by visual inspection.

The range of magnetic field strength of the DC magnetic field source utilized in the present invention is 25 to 300 Oe, with the preferred embodiment being in the range of 35 to 100 Oc.

To ensure detectability of a retained FFB whose magnetic field is in such a direction as not to be detectable by the first sensor, a second sensor is mounted with its plane parallel to the plane of the first sensor, but with its sensitivity axis orthogonal to that of the first sensor and to the applied magnetic field of the DC magnetic field source. In order to discern the existence of an elongated FFB whose orientation just happens to be such that rotation of the eye in one plane (x axis) does not generate a detectable signal, the eye is rotated in the second plane (y axis).

The purpose of the magnetic field of the DC magnetic field source is to magnetize a ferromagnetic foreign body (if present), thereby allowing FFB detection by the sensor apparatus system of the present invention. The present invention, in essence, provides a DC magnetic field source and measurement system in which the applied magnetic field is provided by the magnetic field of the DC magnetic field source, such as a permanent magnet system. Alternatively, a suitable DC magnetic field can be provided by other means, including, but not limited to, a coil with appropriate dimensions and power supply, or a magnetic core with external coil and power supply.

The present invention comprises a sensor system into which the patient's sensed area, such as the eye, is placed within each designated magnetic field strength region, or test zone set point. The sensing system preferably employs magnetoresistive elements, although alternative sensor systems can be employed. Movement of the sensed region, such as of the eye, is employed to allow detection of the induced magnetization of the FFB. For pre-MRI screening of the eye, a moving eye-tracked target is preferably provided to encourage predictable and comfortable movement of the eye for signal detection considerations, i.e., to differentiate the FFB's synchronous signal from background noise. Alternatively, the technician can provide vocal commands to the patient to elicit eye movements, or pre-recorded audio commands can be utilized.

In the preferred embodiment, the sensor utilized is a 4-arm sensor bridge arrangement, which allows for temperature compensation. In order to read the output of the sensor bridge, it is customary to establish a null, which means that the output, i.e., the signal voltage, from the bridge will be adjusted to read at zero. The null is established first without the patient in position, and then, with the patient in position, “fine-tuned” to compensate for diamagnetic considerations of the sensed body area, as described herein. Movement of the sensed region, such as the eye, enables detection of a FFB within the sensed region. Using eye screening as an example, eye movements are graphically documented on a visual display, and the synchronicity of bridge output voltage deviations correlating with eye movements is also graphically documented on a visual display. A two-channel recorder preserves the appropriate test data, including also the name and birth date of the patient, as well as the time of the test. A printer yields a hard copy for the patient's permanent file. A computer may be employed for data processing to make a go/no-go, or yes/no, decision regarding the detection of a FFB using predetermined criteria.

The sensor apparatus of the present invention is best used to achieve the required measurements in, a predetermined fixed alignment relative to the DC magnetic field source's axial magnetic field. Preferably, the axial portion of the DC magnetic field source's magnetic field is employed, as this facilitates the nulling process and avoids the necessity of nulling asymmetrical, off-axis magnetic field components of the instrument's magnet.

An instrument employing a DC magnetic field source can be constructed in which the sensor system moves with respect to the magnetic field, either transversely or axially, or, alternatively, the sensor system can be placed in other areas on the non-axial magnetic field. However, these approaches result in added complexity, because of nulling challenges related to the changing off-axis, and therefore asymmetrical, magnetic field strengths emanating from the DC magnetic field source. Software can be written to compensate for these nulling problems, but the complexity of these embodiments may result in less reliability than the preferred embodiment of a sensor system which has a fixed relationship to the axial component of the instrument's magnetic field.

In addition, it is possible to make measurements not only in the x and y axes, such as by inducing eye movements in horizontal and vertical directions, but also in the z axis, by constructing the present invention in such a manner that the DC magnetic field source moves back and forth in a predefined fashion along its axial magnetic field.

It should be emphasized that the applied DC magnetic field requirement of the present invention must be small enough that, in and of itself, the pre-MRI screening instrument poses minimal risk; yet, the applied magnetic field must be large enough that detectability of a hazardous FFB is achieved. Detectability depends not only upon the magnetic field applied, but also upon the size and shape of the retained FFB, with tiny, FFBs posing a greater detectability challenge, as do spherical particles. In the present invention, the DC magnetic field strength is of sufficient amplitude to magnetize potentially-hazardous FFBs for subsequent detection, while simultaneously posing minimal risk of damage related to the pre-MRI screening procedure itself.

In practice, it is preferred to begin with a lower magnetic field strength test zone set point, and, in serial fashion, move to a higher magnetic field strength test zone set point. One or more of these test zone set points may be utilized in the present invention's pre-MRI screening protocol, and, if desired, alternative test zone set points other than those designated above may be used. Preferentially, the test zone set points lie along the axial component of the DC magnetic field source's magnetic field to readily enable balancing out (nulling) unwanted off-axis magnetic field components of the DC magnetic field source. Employing the axial component of the magnetic field of the DC magnetic field source, then, avoids the necessity of nulling asymmetrical magnetic field components emanating from the DC magnetic field source.

Although a solitary test zone set point can be used, the preferred embodiment of the present invention utilizes a two-staged detection strategy using two test zone set points, such as 35 Oe and 100 Oe.

The primary purpose of this two-staged detection strategy is safety. To elaborate, before the patient is subjected to the albeit very small magnetic field of 100 Oe, the patient is first tested at the extremely low magnetic field of 35 Oe. It is to be noted that a test zone set point of 35 Oe is 428 times less in magnetic-field amplitude than that within the measurement zone of a 1.5 Tesla (15,000 Oe) MRI machine. A 35 Oe test zone set point is also 3 times less in amplitude than the higher test zone set point of 100 Oe. The potential for tissue destruction secondary to induced movement of a significant retained FFB at either 35 Oe or 100 Oe is extremely minimal. Nevertheless, in order that the present invention is rendered as safe as possible, the lower test zone set point is utilized first. If testing is negative at this lower test zone set point, the patient is then tested at the higher test zone set point (which, at 100 Oe, is still 150 times less forceful than the magnetic field strength of a 1.5 T. MRI instrument.)

A second purpose of utilizing a two-staged testing strategy employing two different magnetic field strengths is that this configuration helps to detect not only FFBs which are not previously magnetic, but also FFBs which are in and of themselves magnets, bearing in mind that annealing or pounding upon a ferromagnetic material can result in magnetization of that material. With a two-staged detection strategy, the likelihood of missing a FFB, whether a magnet or not, is greatly diminished. In addition to patient safety, the benefit, then, of employing more than one test zone is for the rare, but yet possible, case in which the FFB is already a magnet. In such an instance, the applied magnetic field provided by the DC magnetic field source at the first designated test zone set point could conceivably be just sufficient to effectively negate the magnetization of the FFB, and detection could fail, in spite of movement of the sensed region. In the next designated test zone set point, however, with a larger applied magnetic field present, detectable magnetization will now be induced, and with movement of the sensed region, a deviation from null will occur. The magnet fragment will then be detected.

Other pairs of test zone set points can be employed, including but not limited to, 50 Oe and 125 Oe, 75 Oe and 200 Oe, or 125 Oe and 300 Oe, and so on, depending upon ongoing clinical research studies. The current preferred embodiment of the present invention is to utilize two test zone set points, and these are 35 Oe and 100 Oe.

Knowing in advance the null setting for each designated test zone set point before the patient is introduced to the sensor system is helpful. Therefore, the null settings for the test zone set point(s) utilized can be established before the patient is positioned for pre-MRI screening, using known test zone set point locations and known null settings corresponding with each test zone set point (step 1. null). As the patient's sensed body region may have diamagnetic susceptibility which modifies the imposed applied magnetic field, however, prior to actual patient measurement, the system is re-nulled (“fine-tuned”) with the patient in position at each designated test zone set point with no motion of the sensed region (step 2. null). Deviations from this final step 2. null by virtue of motion of the sensed region facilitate detection of a retained FFB in the respective sensed region. For instance, eye motion provides detection of a FFB within the eye. Head movement provides detection capability for a FFB within the brain, or within the orbit. An absence of deviation from the final step 2. null in spite of motion of the designated sensed region indicates that no FFB is present in the designated sensed region (with the exception that a previously-magnetized foreign body, such as a magnet or magnet fragment, may escape detection, and this exception is effectively overcome in the preferred embodiment's two-staged testing protocol described above.)

The sensor system is appropriately nulled before the patient is introduced to the sensor system (step 1. null). Although the step 1. null can be omitted, with the technician simply performing the step 2. null and subsequent pre-MRI screening, the inventors recommend that the step 1. null be routinely performed, because this checks the sensor system for proper performance, and, very importantly, confirms a baseline.

Although diamagnetic considerations of the body region being sensed can cause a small deviation from the step 1. null, a significant deviation from this baseline alerts the technician that a FFB may be present, either in the target area to be sensed, or in its adjacent environs. For instance, in the case of eye screening, an immediate and significant deviation, even without induced movement of the eye, could indicate the presence of a FFB within the eye, or within the orbit. In this case, extreme caution must be exercised. In fact, the recommended course of action is to measure the fellow eye and establish its baseline, again without eye movement. If a substantial baseline difference is noted between the two eyes, a FFB is strongly suspected within the eye or in the adjacent tissue of the eye (such as the orbit).

After establishing the step 1. null, the patient is then physically presented to the sensor system, and the sensor system is properly positioned to sense the desired body region of the patient, such as the eye. Because of diamagnetic considerations of the body region being sensed, it is necessary that the sensor system be “fine-tuned” (step 2. null), and, after such step 2. nulling, the patient is instructed to move the region being sensed (such as the eye, or the head). A deviation from this step 2. null indicates the presence of a ferromagnetic foreign body within the sensed region, in which instance the MRI procedure is aborted. An x-ray should then be considered to document the presence of a retained FFB, keeping in mind the risk of ionizing radiation from x-rays, which could be contraindicated in certain instances, such as with a pregnant woman.

In the preferred ferromagnetic foreign body detection method of the present invention, the magnet and the sensing apparatus system are attached permanently in a fixed relationship to the instrument. Designated test-zone-set-point locations, preferably along the magnetic field axis, each with a corresponding null setting, are established before introducing the patient to the sensor system. The null settings noted at these test-zone-set-point locations are duly recorded and reconfirmed for safety reasons before each patient is tested.

A typical protocol is as follows:

1. Before placing the patient in position to be examined, establish the null for the test zone set point correlating with the lower designated magnetic field strength, such as 35 Oe, and then for the higher designated magnetic field strength, such as 100 Oe.

(In a simplified alternative embodiment, a solitary test zone set point with its corresponding null point is utilized at the distance from the magnet correlating with a designated magnetic field strength, such as 100 Oe. A potential problem with a one-test-zone-set-point system, however, is that a magnet fragment may escape detection. Magnetic foreign bodies are believed to be quite rare. Nevertheless, this problem is circumvented by using more than one test zone set points.)

2. Introduce the patient to the instrument, such that the sensor is appropriately positioned to measure, at the weakest designated test zone set point, the body region of the patient to be sensed, such as the patient's eye.

3. Now that the patient is properly positioned for pre-MRI screening at the first test zone set point corresponding with the weakest designated magnetic field strength, re-null the sensor apparatus (step 2. null), to “fine-tune” the sensor. This re-nulling is performed without movement of the body region to be sensed. Re-nulling is necessary because the body region to be inspected for an FFB has diamagnetic susceptibility which may modify the imposed applied magnetic field, so that it is necessary to re-establish the null (step 2. null, i.e., “fine-tuning” of the step 1. null) before commencing movement of the body region and subsequent interrogation of the sensor apparatus.

4. The patient is then instructed to move the body region to be sensed, preferably in a controlled fashion. For eye detection, eye movements are horizontal and vertical along x and y axes, and it is desirable to have a target which can be readily seen and thereby tracked by the patient. Alternatively, vocal commands may be employed, which can be pre-recorded for convenience. Controlled movements enhance coherency, and thereby detectability, of the signal emanating from the FFB which has been magnetized by the instrument's magnet, said movement distinguishing the FFB from background noise.

5. The sensor apparatus is then interrogated while the sensed body area containing a possible FFB is moved. This movement provides detectability of the FFB, and such detection will be indicated by a measurement reading deviating from the step 2. null.

6. If a ferromagnetic foreign body is detected, abort the MRI procedure. If it is deemed medically imperative that the patient receive the MRI, send the patient for confirmatory testing, such as by x-ray.

7. In the preferred embodiment utilizing two test zone set points, if no ferromagnetic foreign body is detected at the first test zone set point, move the patient toward the instrument's DC magnetic field source to the second test zone set point, which has a greater magnetic field strength than the first test zone set point. The preferred embodiment for the first test zone set point is 35 Oe, and, for the second test zone set point, 100 Oe.

8. Next, appropriately set the sensor apparatus according to predetermined measurements for this second test zone set point by using the pre-established step 1. null for this location.

9. With the patient in place for appropriate FFB sensing, perform the step 2. null (“fine-tuning”) at this second test zone set point. Then, instruct the patient to move the body region to be sensed, preferably in a controlled fashion. Interrogate the sensor apparatus system. A ferromagnetic foreign body will be detected by a reading deviating from null, induced by movement of the magnetized FFB.

10. If a ferromagnetic foreign body is detected at this second test zone set point, abort the MRI procedure. The patient is then referred for appropriate confirmatory testing.

11. The instrument may employ more than two test zone set points, with each test zone set point increasing serially in magnetic field strength. For instance, if a third test zone set point were desired, this could be at a magnetic strength of 200 Oe, and a fourth could be at 700-1000 Oe. Increasing the number of test zone set points adds to the complexity of the instrument, as well as to the time required for patient screening.

12. Serial interrogation as defined herein proceeds until the designated test zone set point with the greatest magnetic field strength is interrogated, aborting the MRI if a deviation form the step 2. null is noted at any test zone set point. After all desired test zone set points are interrogated and noted to be negative for the detection of a ferromagnetic foreign body, the patient may then undergo magnetic resonance testing safely, with a very high degree of confidence.

It should be emphasized that, although a one test-zone-set-point system is simple, efficient, and time saving, to increase detection probability in the event that the FFB in question is a magnet, a two-or-more test-zone-set-point system provides more assurance that no FFB is present, be the FFB a magnet, or not.

The present invention is aptly suited to telemedicine, and the preferred telemedicine vehicle is the Internet. Artificial intelligence modalities, including neural net and other expert systems, can also be used, providing instantaneous autointerpretation of test results. Provision is made for providing real-time interactive feedback between the remote instrument and a central computer processing station, helping to ensure the appropriate functioning of the instrument, the use of proper test protocols, and reliable data acquisition.

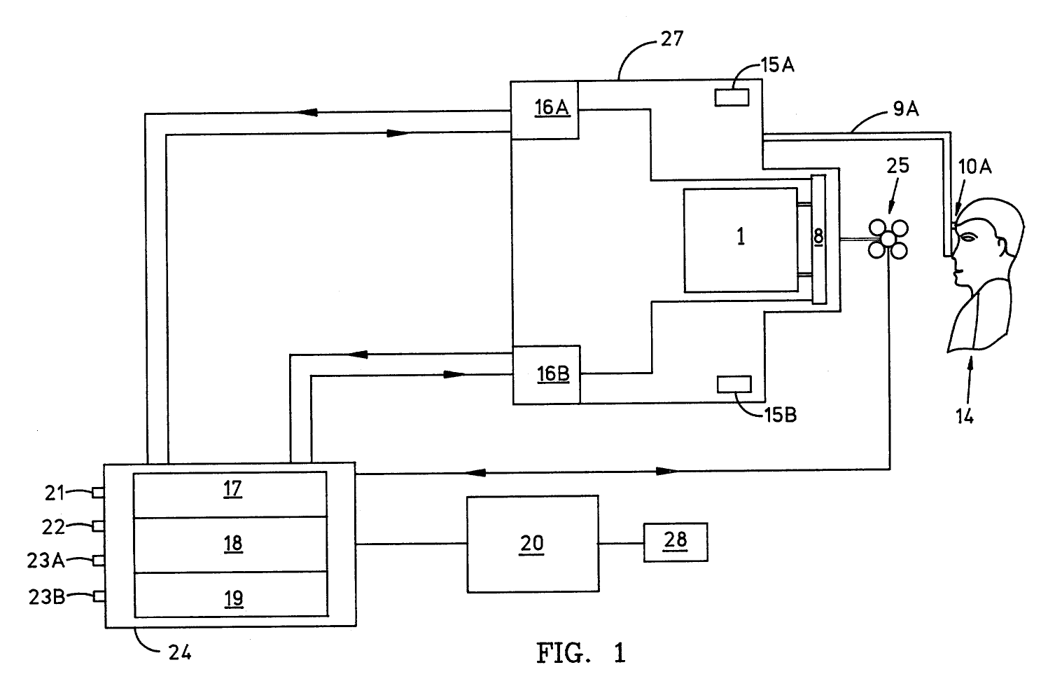

FIG. 1 demonstrates a patient 14 in place for pre-MRI screening according to the present invention. The eye is properly positioned at the designated distance from the DC magnetic field source 1, with the distance being determined by an eye position register 9A.

Measurements are made preferentially along the axial component of the magnetic field of the DC magnetic field source 1, beginning with the weakest magnetic field strength, and proceeding in serial fashion to distances from the DC magnetic field source 1 corresponding with increasingly stronger magnetic field strengths. The preferred embodiment utilizes measurements at distances corresponding with magnetic field strengths of 35 Oe and 100 Oe.

Provision is made to discontinue the measurements whenever the eye is not in the proper position, using a contact button 10A, or similar apparatus. If the patient 14 inadvertently moves away from the contact button 10A, the instrument registers an error reading, and the measurements in question must be repeated with the patient in the proper position.

The sensor apparatus system 8, which has been nulled before the patient 14 is introduced to the instrument (step 1. null), is now renulled after the patient 14 is properly positioned for pre-MRI screening (step 2. null). A small difference between the step 1. null and the step 2. null is frequently noted, because of diamagnetic considerations of the sensed region itself.

The patient 14 is then instructed to move the sensed body region, such as the eye, preferably in a controlled fashion. During this movement, the sensor apparatus system 8 is interrogated. Eye movements are preferentially in the horizontal (x) and vertical (y) axes, and these eye movements are directed by a moving eye-tracked target 25, such as a light-emitting diode (LED) moving display. Alternatively, the technician can provide vocal commands to the patient to elicit controlled eye movements, or pre-recorded audio commands can be utilized.

A ferromagnetic foreign body (FFB) will be detected by a measurement reading deviating from the step 2. null, during eye movement. An intraocular FFB will show deviations from the step 2. null which are synchronous with the eye movements. If a ferromagnetic foreign body is detected, abort the MRI procedure. If no FFB is detected at the distance from the DC magnetic field source 1 corresponding with the weakest magnetic field strength, the eye is screened next at a distance closer to the DC magnetic field source 1, and, in serial fashion, at all other desired distances.

Supplementary magnetic field sources 15A, 15B such as magnets or coil apparati, are employed to null environmentally existing and unwanted components of DC magnetic fields which may also lie in the plane of the sensor apparatus system 8. This may be accomplished using the main DC magnetic field source 1, but more readily by the use of smaller, supplementary magnets, or other compensating ferromagnetic materials, such as coils.

The sensor apparatus system 8 feeds signals generated in response to the sensed magnetic field to amplification/signal conditioning modules 16A, 16B which in turn are connected to a control console module 24 containing a DC power supply 19, a display 17, and a recorder 18.

The sensor apparatus system 8, the DC magnetic field source 1, the supplementary magnetic field sources 15A, 15B, and the amplification/signal conditioning modules 16A, 16B are preferably housed in a data acquisition module 27, which is interconnected via external cables to the control console module 24.

Signals received by the sensor apparatus system 8 associated with eye movement in the horizontal (x) axis are sent to one amplification/signal conditioning module 16A, and signals received by the sensor apparatus system 8 associated with eye movement in the vertical (y) axis are sent to another amplification/signal conditioning module 16B. Provision is made for resetting the sensor apparatus system 8 to its most sensitive null field position, called “conditioning,” and is preferentially done automatically by the circuitry, although a manual reset button may also be used for this purpose. Interconnecting circuitry between the data acquisition module 27 and the control console module 24 allows for signal routing between the modules, with sensed information flowing from the data acquisition module 27 to the control console module 24, which generates feedback to the data acquisition module 27 for coordination of instrument parameters and proper operation.

Interconnecting circuitry to and from the control console module 24 and the eye-tracked target 25 coordinate the eye movements, the graphic display thereof, and the synchronicity, or lack thereof, with the sensed magnetic field received by the sensor apparatus system 8.

Adjustment knobs 21, 22 located on the control console module 24 establish nulls, and other adjustment knobs 23A, 23B also located on the control console module 24, are utilized for gross sensitivity and fine sensitivity tuning of the sensor apparatus system 8. Other appropriate adjustment knobs, dials, and controls are employed, as desired. The data which are displayed may also be processed by a computer means 20 to make a go/no-go, or yes/no, decision regarding the detection of a FFB, using pre-determined criteria. A printer 28 preserves a hard copy of the data.

The recorder 18 is multi-channel, recording, on one or more channels, eye movements directed by the eye-tracked target 25 or by verbal commands, and, on one or more additional channels, the sensed magnetic field transmitted from the sensor apparatus system 8.

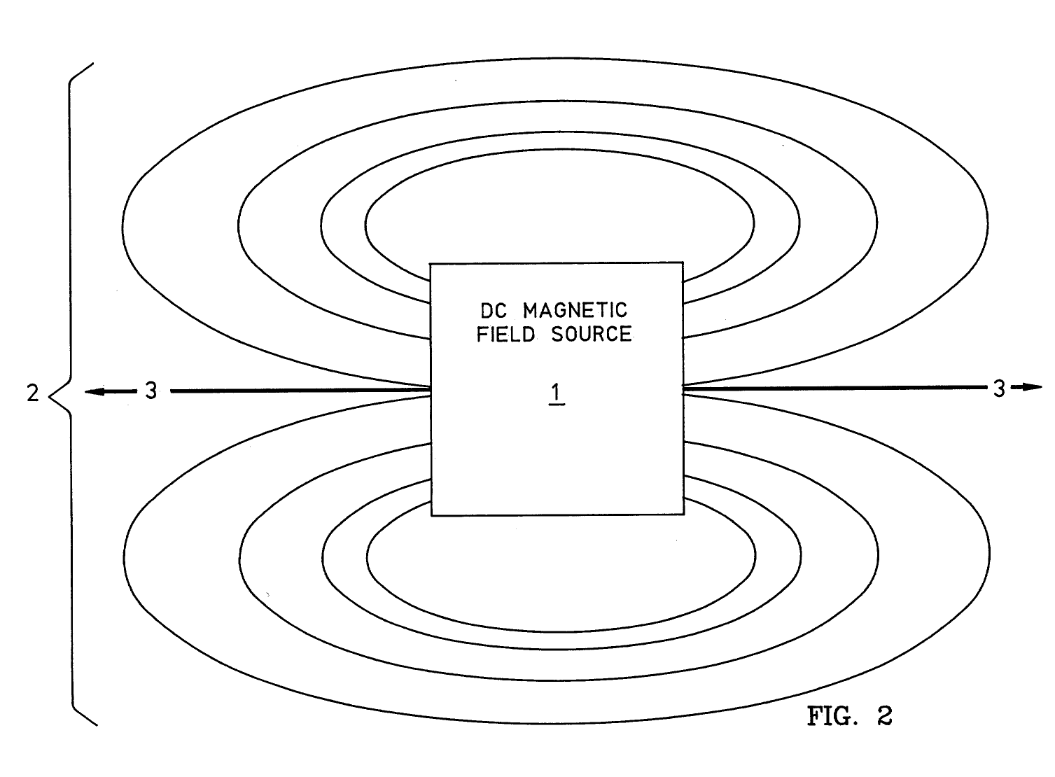

FIG. 2 depicts the magnetic field 2 of the DC magnetic field source 1 of the present invention. The axial component 3 of the magnetic field 2 is depicted. A permanent magnet or other source of DC magnetic field may be utilized, such as a coil with appropriate dimensions and power supply, or a magnetic core with an external coil and power supply.

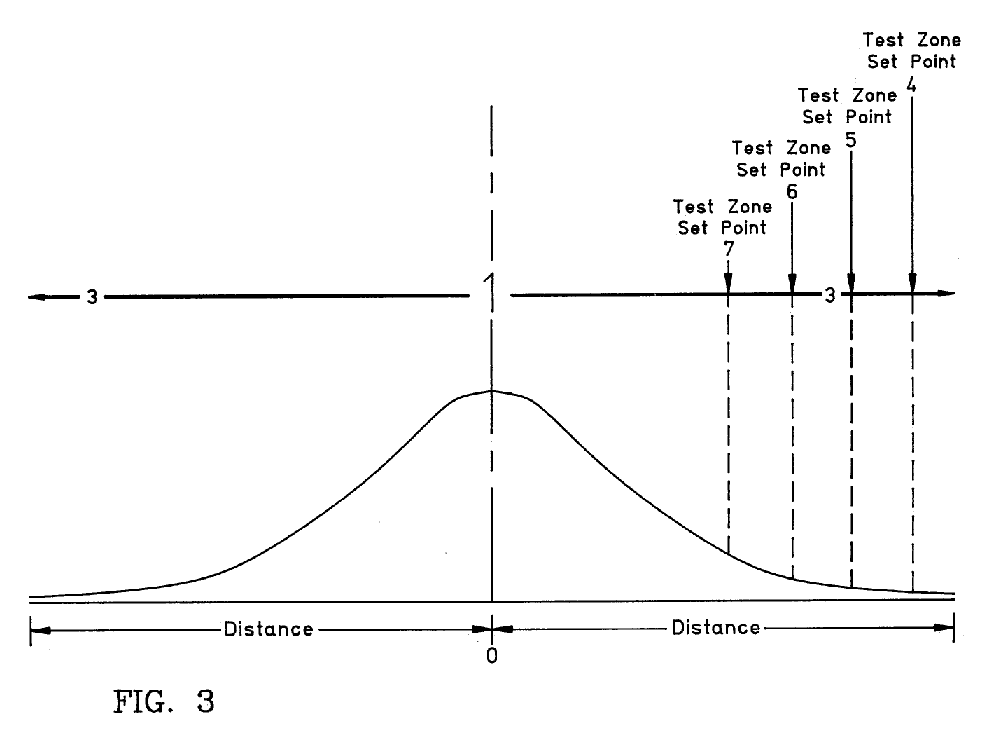

FIG. 3 depicts the diminution in strength of the magnetic field as the distance from the DC magnetic field source 1 increases. Representative first and second “test zone set points” 4, 5 utilized for pre-MRI screening are shown, and, if more than two test zone set points are desired, additional test zone test points 6, 7 can be designated.

The test zone set points 4, 5, 6, 7, and others as desired, each with a corresponding null position, are established and documented (step 1. null). All test zone set points lie preferably along the axial component 3 of the magnetic field, to readily enable balancing out (nulling) unwanted asymmetrical magnetic field components of the DC magnetic field source 1.

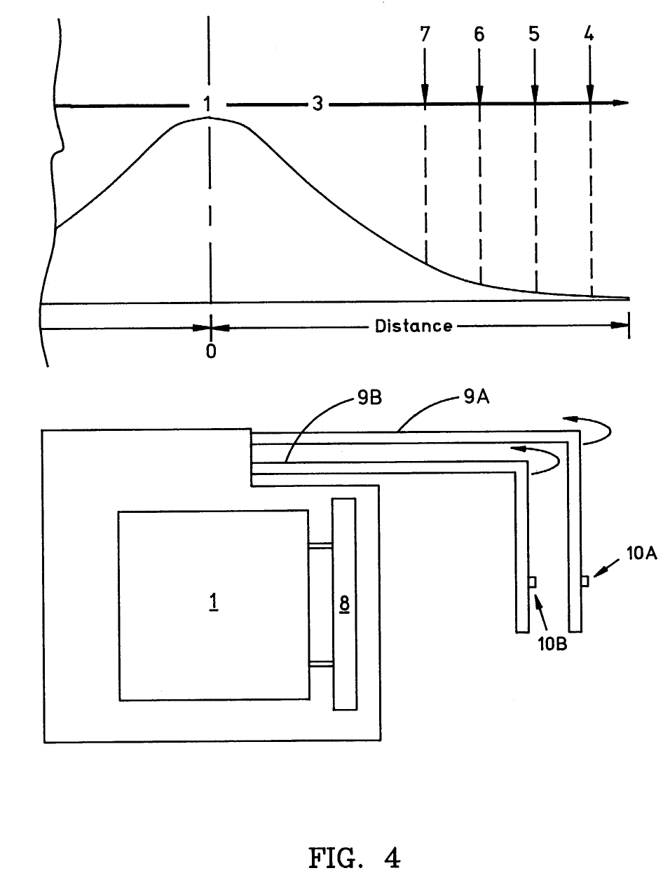

FIG. 4 shows the DC magnetic field source 1 for the present invention's pre-MRI screening instrument, with the attached sensor apparatus system 8. Appropriate distances from the DC magnetic field source 1 are pre-determined by eye position registers 9A, 9B which may be releasably adjusted, as desired, to move closer to or farther away from the DC magnetic field source 1. Measurements are made, preferably, along the axial component 3 of the magnetic field of the DC magnetic field source 1, beginning with the weakest magnetic field strength, corresponding with the first test zone set point 4. The preferred embodiment utilizes two test zone set points 4, 5 corresponding with magnetic field strengths of 35 Oe and 100 Oe.

Provision is made to discontinue the measurement whenever the eye is not in the proper position. Although other means may be employed, a contact button 10A, 10B is a simple solution which effectively accomplishes this purpose. For instance, if a patient inadvertently moves away from the contact button 10A, 10B, the instrument registers an error reading, and the measurements in question must be repeated with the patient in the proper position. The use of this fail-safe mechanism increases the reliability of the system.

An outer eye position register 9A positions the eye farther away from the DC magnetic field source 1, subjecting the eye to less magnetic field strength than when an inner eye position register 9B is utilized.

Therefore, when performing pre-MRI screening using the outer eye position register 9A, which positions the eye so that it corresponds with the first test zone set point 4, the eye is subjected to a lower magnetic field strength. Then, when utilizing the inner eye position register 9B, which positions the eye closer to the DC magnetic field source 1, corresponding with the second test zone set point 5, the eye now is subjected to a higher magnetic field strength than that encountered with the outer eye position register 9A. Additional eye position registers (not shown) may be utilized which correspond with the third and fourth test zone set points 6, 7 and others, if desired, with the third test zone set point 6 manifesting a higher magnetic field strength than the second test zone set point 5, and the fourth test zone set point 7 manifesting a higher magnetic field strength than the third test zone set point 6. The test should begin with the designated test zone set point corresponding with the lowest magnetic field strength, and sequentially proceed at designated test zone set points with increasingly higher magnetic field strengths.

The eye position registers 9A, 9B, by moving along the axial component 3 of the magnetic field, lend themselves to maintaining the desired orientation for the sensor apparatus system 8 and the patient.

The DC magnetic field source 1 supplies the applied magnetic field to magnetize a ferromagnetic foreign body, if present, and thus allow its detection by the sensor apparatus system 8.

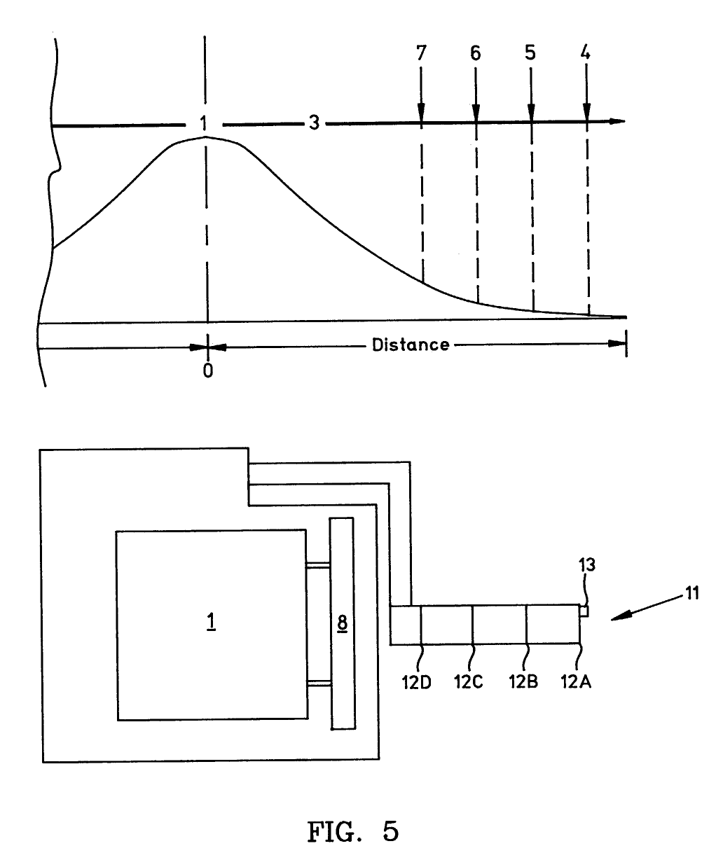

As shown in FIG. 5, alternate means may be utilized for providing the desired distances of the eye, or other sensed body region, from the DC magnetic field source 1. One such embodiment is a proboscis arrangement 11, which can be sequentially adjusted to designated pre-set distances 12A, 12B, 12C, 12D, and others, as desired. One way for accomplishing adjustments of the proboscis arrangement 11 is to employ a collapsible column.

Provision is made on the proboscis arrangement 11 for a fail-safe mechanism which discontinues measurements and registers an error reading whenever the patient is not in the proper screening position. A contact button 13, or other means to ensure proper patient positioning, may be employed for this purpose. The measurements must then be repeated with the maintenance of appropriate physical continuity between the patient and the contact button 13.

In the two-staged preferred embodiment of the method of the present invention, measurements commence utilizing a first pre-set distance 12A which corresponds with a distance from the magnetic field source 1 which manifests the lowest desired magnetic field strength. Measurements then proceed at a second pre-set distance 12B which manifests a higher magnetic field strength than that corresponding with the first pre-set distance 12A. If further measurements are warranted according to the test protocol, the proboscis arrangement 11 may be adjusted accordingly to third and fourth pre-set distances 12C, 12D, and other pre-set distances, as desired.

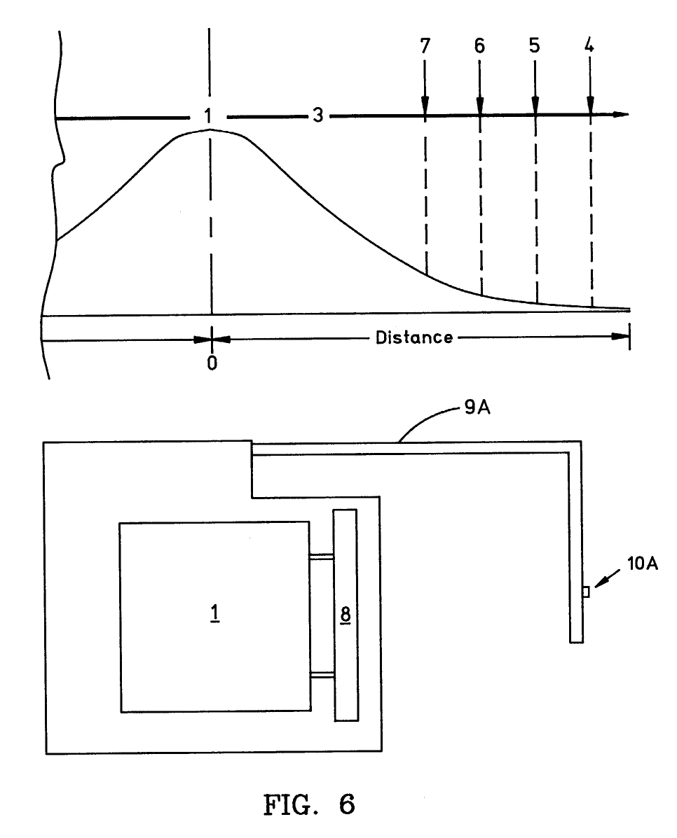

FIG. 6 depicts part of the pre-MRI screening instrument without the patient in position. With the sensor apparatus system 8 and the DC magnetic field source 1 in a fixed, rigid spatial relationship, the outer eye position register 9A is positioned such that the measurement distance of the eye from the DC magnetic field source 1 corresponds with the weakest strength test zone set point 4 along the axial component 3 of the magnetic field.

The sensor apparatus system 8 is then appropriated nulled, and the settings for null are documented (step 1. null).

Using these step 1. null settings for reference, it becomes easier to perform the step 2. null (“fine-tuning”) procedure after the patient is introduced to the pre-MRI screening instrument. The step 2. null (“fine-tuning”), as described herein below, is obligatory to compensate for diamagnetic considerations, which can vary from patient to patient. Deviations from the step 2. null setting induced by movement of the sensed body region indicate the presence of a FFB.

In a simplified embodiment, a solitary test zone set point with its corresponding null point can be utilized at a distance from the DC magnetic field source 1 which correlates to a designated magnetic field, such as 100 Oe.

It is preferred to employ more than one test zone set point. Representative test zone set points are, by way of example, 35 Oe, 100 Oe, 300 Oe, and 1,000 Oe, and even higher, if desired. The preferred embodiment utilizes two test zone set points, 35 Oe and 100 Oe. Whatever test zone set points are used, it is preferred to establish a corresponding step 1. null reference setting for each designated test zone set point, during the initial instrument setup before patient introduction.

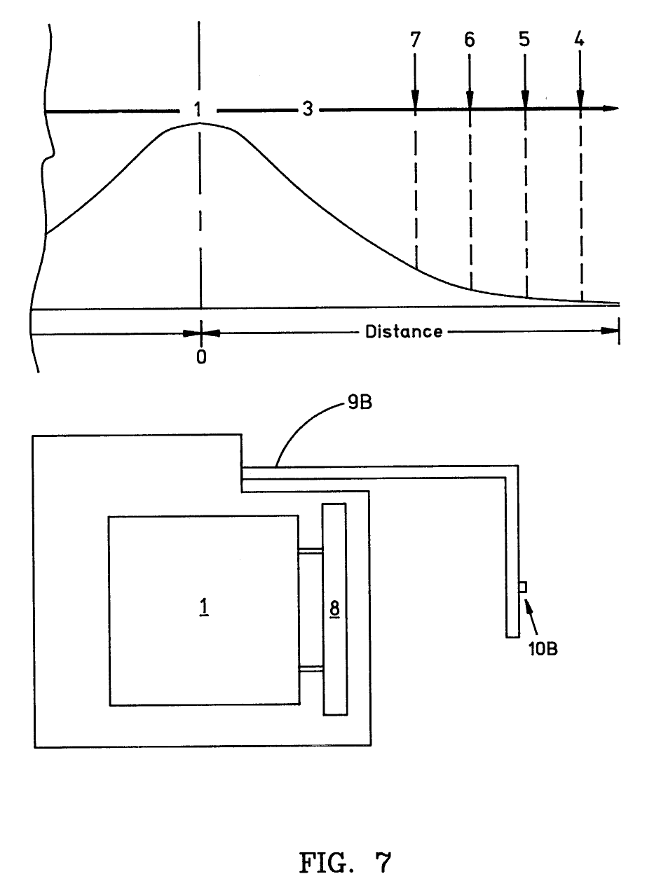

FIG. 7 depicts part of the pre-MRI screening instrument without the patient. With the sensor apparatus system 8 and the DC magnetic field source 1 in a fixed, rigid spatial relationship, the inner eye position register 9B is positioned along the axial component 3 of the magnetic field such that the measurement distance for the eye from the DC magnetic field source 1 corresponds with the next-in-sequence test zone set point 5, which has a higher designated magnetic field strength than the first test zone set point 4. The sensor apparatus system 8 is then appropriately nulled (step 1. null).

Although a two test-zone-set-point instrument is preferred, such as one utilizing the first and second test zone set points 4, 5 corresponding preferably with 35 Oe and 100 Oe, respectively, additional test zone set points may be utilized, such as the third and fourth test zone set points 6, 7, and others, if desired. These lie along the axial component 3 of the magnetic field of the DC magnetic field source 1. As each test zone set point becomes closer in physical proximity to the DC magnetic field source 1, its correlating magnetic field strength increases.

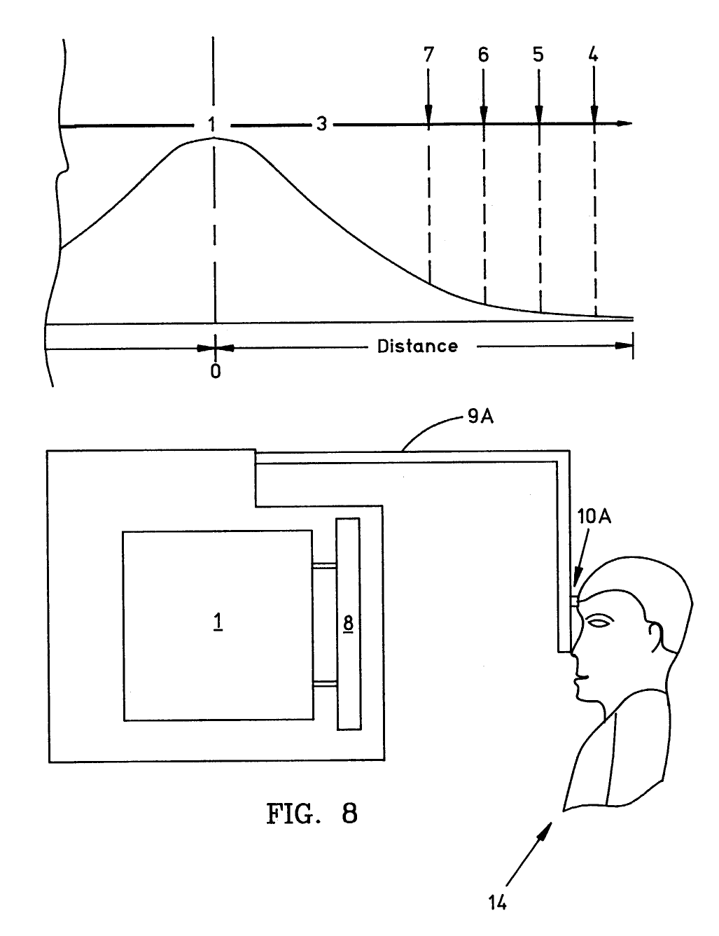

In FIG. 8, the patient 14 is introduced to the instrument in the appropriate position to have the desired body region, such as the eye, interrogated by the sensor apparatus system 8. The eye of the patient 14 is juxtapositioned to the eye position register 9A at the appropriate distance corresponding with the first test zone set point 4. At this location, the eye is subjected to the weakest desired magnetic field, such as 35 Oe.

The sensor apparatus system 8 is set according to predetermined measurements (step 1. null) for the first test zone set point 4, which were made as shown in FIG. 6. The sensors generally embody a reset provision. This provision enables the desirable, non-saturated sensing condition to be re-established automatically during use. The contact button 10A ensures the maintenance of proper patient positioning during pre-MRI screening measurements.

“Fine-tuning” of the null point (step 2. null) is performed with the sensed body region, such as the eye, in position for FFB detection before movement of the sensed body region takes place. A small difference between the step 1. null and the step 2. null is frequently noted, because of diamagnetic considerations of the sensed region itself.

A significant difference between the step 1. null and the step 2. null, however, raises a warning flag that a FFB may be present in the sensed body region, or its environs. In this event, extreme caution must be exercised. For eye screening, if a significant difference is noted between the step 1. null and the step 2. null for one eye, it is recommended to ascertain whether or not a similar difference is noted for the fellow eye. If a similar difference is not noted for the fellow eye, the first eye (or the orbit) is highly suspicious for the presence of a FFB, and no further exposure to the magnetic field should be allowed. The patient should be considered for x-ray radiography before allowing magnetic resonance imaging. Note that the measurements described in this step are all without movement of the sensed body region, such as the eye.

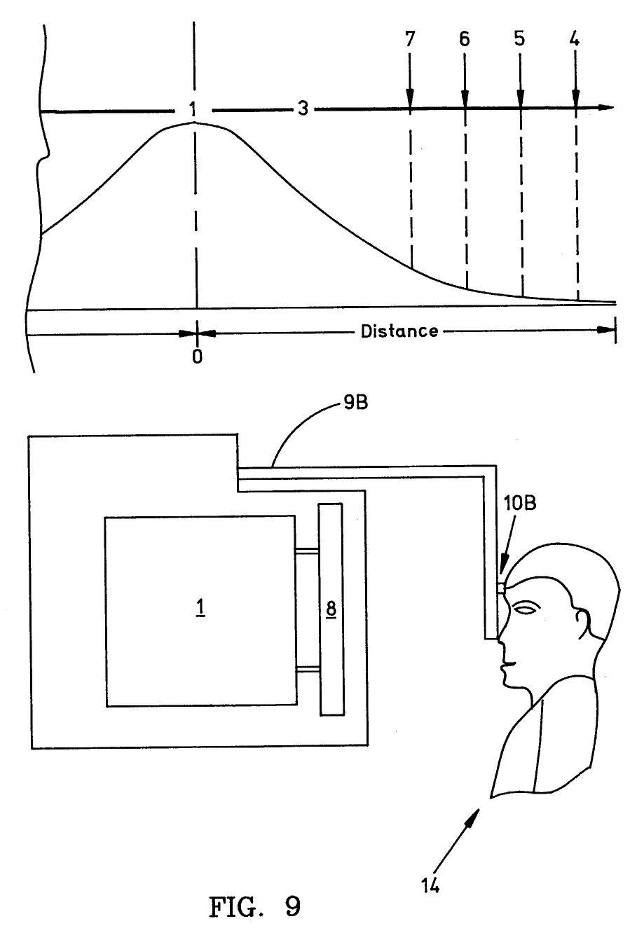

After attaining the step 2. null, if the step 1. and step 2. nulls are reasonably close, however, the patient 14 is then instructed to move the sensed body region, such as the eye. During this movement, the sensor apparatus system 8 is interrogated. A ferromagnetic foreign body will be detected by a measurement reading deviating from the step 2. null. If a ferromagnetic foreign body is detected, abort the MRI procedure. If no FFB is detected at the weakest magnetic field strength test zone set point 4, the eye position register is moved along the axial component 3 of the magnetic field to the next designated test zone set point 5, as shown in FIGS. 7 and 9.

In the embodiment employing only a solitary test zone set point for the MRI pre-screening procedure, such as, for example, the 100 Oe test zone set point, if no FFB is detected at this test zone set point, the patient 14 can then be advanced into the MRI instrument and imaging can proceed.

The potential problem with a solitary test-zone-set-point detection system in the presence of a foreign body which is in and of itself a magnet or magnet fragment has been described within this application. The two (or more) test-zone-set-point method provides an effective solution to this potential problem, as well as being a safer method, because the first test zone set point in this embodiment subjects the sensed region of the patient 14 to a significantly smaller magnetic field than that which would be employed in a solitary test-zone-set-point method. For instance, a two test-zone-set-point method may employ test zone set points of 35 Oe and 100 Oe, while a solitary test-zone-set-point method may employ a test zone set point of only 100 Oe, which is 3 times higher than the initial test zone set point of the preferred two test-zone-set-point method.

An interesting situation in eye pre-MRI screening is that in which a large difference is present between the step 1. null (i.e., that established before patient introduction) and the step 2. null (i.e., with the patient in position), but in which this difference is not synchronous with eye movements. This situation could indicate that a FFB is present in the orbit, hence explaining lack of synchronicity with eye movements. If the difference is synchronous with eye movements, the FFB is intraocular. If non-synchronous, a potential harmful FFB may be lodged in the orbit. In any case, a large difference between the step 1. null and the step 2. null warrants further investigation. Such a patient must not undergo magnetic resonance imaging without further ancillary testing, such as by x-ray radiography.

In FIG. 9, the patient 14 is introduced to the instrument in the appropriate position to have the desired body region, such as the eye, sensed by the sensor apparatus system 8. For eye screening, the eye of the patient 14 is juxtapositioned to the eye position register 9B at the appropriate distance corresponding with the next-in-sequence test zone set point 5. At this location, the eye is subjected to a larger magnetic field, such as 100 Oe, than that encountered at the previous test zone set point, i.e., the first test zone set point 4.

The sensor apparatus system 8 is set according to predetermined measurements (step 1. null) for the second test zone set point 5, which were made as shown in FIG. 7. “Fine-tuning” of the null point (step 2. null) is performed without movement of the sensed body region, such as the eye. The contact button 10B ensures the maintenance of proper patient positioning during pre-MRI screening measurements. The test continues, as described in the discussion of FIG. 8, but now at this new higher magnetic field strength.

If desired, additional measurements may be made at additional test zone set points at sequentially increasing magnetic field strengths, following the protocol established above. Measurements are made at each test zone set point, first without movement of the sensed region, and then with movement of the sensed region. A ferromagnetic foreign body will be detected by a measurement reading deviating from the step 2. null. If a ferromagnetic foreign body is detected, the MRI procedure must be aborted, and the patient should be removed away from the instrument's magnetic field, preferably in a slow and controlled manner to avoid dislodging the FFB, or otherwise causing it to move. Synchronicity of eye movement with deviations from the step 2. null is confirmatory that a FFB is present within the eye. These data are graphically documented. Computer processing may be employed to make a go/no-go, or yes/no, decision regarding the detection of a FFB using pre-determined criteria, and hard copies are preserved by the printer.

After all desired test zone set points are interrogated in the serial fashion described herein and no FFB is detected, the patient 14 is introduced into the MRI instrument, and magnetic resonance imaging commences.

While the particular invention as herein shown and disclosed in detail is fully capable of obtaining the objects and providing the advantages hereinbefore stated, it is to be understood that this disclosure is merely illustrative of the presently preferred embodiments of the invention and that no limitations are intended other than as described in the appended claims.

We claim:

1. A method for screening a patient for the presence of a ferromagnetic foreign body, comprising:
providing a DC magnetic field source, a magnetic field sensor, and a patient positioning apparatus;
generating a magnetic field with said DC magnetic field source;
positioning a portion of a patient at a first test zone set point along the axis of said magnetic field, with said patient positioning apparatus;
nulling the output signal of said sensor with said patient positioning apparatus at said first test zone set point, with said patient present;
orienting said portion of said patient at a plurality of orientations, at said first test zone set point; and
detecting any change in said output signal of said sensor as said portion of said patient changes orientation at said first test zone set point.

2. The method recited in claim 1, further comprising:
positioning said patient positioning apparatus at said first test zone set point; and
nulling said output signal of said sensor with said patient positioning apparatus at said first test zone set point, without a patient present.

3. The method recited in claim 1, further comprising:
positioning said portion of said patient at a second test zone set point along the axis of said magnetic field, closer to said DC magnetic field source than said first test zone set point, with said patient positioning apparatus;
nulling said output signal of said sensor with said patient positioning apparatus at said second test zone set point, with said patient present;
orienting said portion of said patient at a plurality of orientations, at said second test zone set point; and
detecting any change in said output signal of said sensor as said portion of said patient changes orientation at said second test zone set point.

4. The method recited in claim 3, further comprising:
sequentially positioning said patient positioning apparatus at each said test zone set point; and
sequentially nulling said output signal of said sensor with said patient positioning apparatus at each said test zone set point, without a patient present.

5. The method recited in claim 1, further comprising maintaining a fixed spatial relationship between said DC magnetic field source and said magnetic sensor.

6. The method recited in claim 1, further comprising limiting said DC magnetic field to a strength no greater than 300 Oe.

7. The method recited in claim 1, wherein said positioning a portion of a patient at a first test zone set point comprises positioning an eye of said patient at said first test zone set point.

8. A method for screening a patient for the presence of a ferromagnetic foreign body, comprising:
providing a DC magnetic field source, a magnetic field sensor, and a patient positioning apparatus;
generating a magnetic field with said DC magnetic field source;
nulling the output signal of said sensor, without a patient present;
positioning a portion of a patient at a first test zone set point along the axis of said magnetic field, with said patient positioning apparatus;
nulling the output signal of said sensor with said patient positioning apparatus at said first test zone set point, with said patient present;
orienting said portion of said patient at a plurality of orientations, at said first test zone set point;
detecting any change in said output signal of said sensor as said portion of said patient changes orientation at said first test zone set point;
positioning said portion of said patient at a second test zone set point along the axis of said magnetic field, closer to said DC magnetic field source than said first test zone set point, with said patient positioning apparatus;
nulling said output signal of said sensor with said patient positioning apparatus at said second test zone set point, with said patient present;
orienting said portion of said patient at a plurality of orientations, at said second test zone set point; and
detecting any change in said output signal of said sensor as said portion of said patient changes orientation at said second test zone set point.

9. The method recited in claim 8, wherein said nulling the output signal of said sensor, without a patient present, further comprises:
positioning said patient positioning apparatus at said first test zone set point;
nulling the output signal of said sensor with said patient positioning apparatus at said first test zone set point, without a patient present;
positioning said patient positioning apparatus at said second test zone set point; and
nulling said output signal of said sensor with said patient positioning apparatus at said second test zone set point, without a patient present.

10. The method recited in claim 8, further comprising maintaining a fixed spatial relationship between said DC magnetic field source and said magnetic sensor.

11. The method recited in claim 8, further comprising limiting said DC magnetic field to a strength no greater than 300 Oe.

12. The method recited in claim 8, wherein said positioning a portion of a patient at a selected said test zone set point comprises positioning an eye of said patient at said selected test zone set point.

13. An apparatus for screening a patient for the presence of a ferromagnetic foreign body, comprising:
a DC magnetic field source adapted to generate a magnetic field;
a magnetic field sensor;
a patient positioning apparatus adapted to selectively position a portion of a patient at a point on the axis of said magnetic field;
means for nulling the output signal of said sensor; and
means for detecting any change in said output signal of said sensor.

14. The apparatus recited in claim 13, further comprising computer means for analyzing said output signal of said sensor to determine the presence of a ferromagnetic foreign body in said portion of said patient.

15. The apparatus recited in claim 13, further comprising means for changing the orientation of said portion of said patient at said point on the axis of said magnetic field.

16. The apparatus recited in claim 13, further comprising a sensor for detecting movement of said portion of said patient away from said point on the axis of said magnetic field.

17. The apparatus recited in claim 13, wherein said patient positioning apparatus is further adapted to selectively position an eye of said patient at said point on the axis of said magnetic field.

18. The apparatus recited in claim 13, wherein said patient positioning apparatus is further adapted to sequentially position said portion of said patient at a plurality of selected points along the axis of said magnetic field.

19. The apparatus recited in claim 18, further comprising means for changing the orientation of said portion of said patient at each said selected point along the axis of said magnetic field.

20. The apparatus recited in claim 18, further comprising a sensor for detecting movement of said portion of said patient away from each said selected point along the axis of said magnetic field.

21. The apparatus recited in claim 18, wherein said patient positioning apparatus is further adapted to sequentially position an eye of said patient at each said selected point along the axis of said magnetic field.
